(19) United States
(12) Patent Application Publication
(10) Pub. No.: US 2023/0055635 A1
(43) Pub. Date: Feb. 23, 2023

(54) Organic Acid Production Process Using Aspergillus Strains Consuming Methanol

(71) Applicant: Myongji Univ. Industry & Academia Cooperation Fndn, Gyeonggi-do (KR)

(72) Inventors: Shin Sik CHOI, Seoul (KR); Ji Eun LEE, Gyeonggi-do (KR)

(21) Appl. No.: 17/788,890

(22) PCT Filed: Oct. 20, 2020
(86) PCT No.: PCT/KR2020/014290

§ 371 Date: Aug. 31, 2022

(30) Foreign Application Priority Data
Dec. 27, 2019 (KR) 10-2019-0176044

(51) Int. Cl. C12N 1/14; C12P 7/46

(52) U.S. Cl. CPC C12N 1/14; C12P 7/46; C12N 2500/34; C12N 2500/14; C12N 2500/16

(57) ABSTRACT

The present disclosure relates to a production medium for microorganisms converting methanol to an organic acid and a culture process, wherein the converted organic acid is oxalic acid, and the production medium for microorganisms comprises 1 to 5% of methanol, 1 to 5% of xylose, and 0.01 to 0.05% of calcium chloride relative to 1 L of the total medium, and further comprises potassium dihydrogen phosphate (KH₂PO₄), ammonium sulfate ((NH₄)₂SO₄), magnesium sulfate (MgSO₄), iron sulfate (FeSO₄), manganese sulfate (MnSO₄), zinc sulfate (ZnSO₄), or boric acid (H₃BO₃). According to the present disclosure, provided is an organic acid production process using microorganisms of the genus Aspergillus (Aspergillus. sp), which enables a high-throughput production of high-value-added value organic acids such as oxalic acid by utilizing methanol obtained as a product from refining Cl gas such as methane.

### TECHNICAL FIELD

The present disclosure relates to an organic acid production process using microorganisms. More specifically, the present disclosure relates to a production process for increasing the existing biological methanol-organic acid conversion rate through evolutionary mutagenesis of microorganisms using methanol as a carbon source.

### BACKGROUND ART

As a result of analyzing the technology level evaluation for 120 national strategic technologies in the 2012 Technology Level Evaluation (Ministry of Science, ICT and Future Planning, February 2013) and Korea Industrial Technology Association survey results, it was analyzed that the level of domestic technology such as bioenergy technology, useful waste resource recycling technology, and eco-friendly biomaterial technology was lower than that of countries with the highest technology. Countries around the world are increasingly interested in biofuels, and countries need to prepare for the era of gas chemistry.

According to a review paper published in Current Opinion in Biotechnology in 2018 by Bennett et al., methane and methanol were biologically converted into fuels and compounds by methylotrophs microorganisms. Cell-free metabolic engineering techniques have been widely used in the biological conversion of methanol and at the core of such methods were the MDHs enzyme, the methanol concentration cycle (MCC), and the nonoxidative glycolis (NOG) pathway.

As this interest grows, research is being conducted on the technology to convert methane, which is considered a representative greenhouse gas, into organic acid. Therefore, a lot of interest and research is underway in the production process of culturing microorganisms strains at high concentrations using low-purity methanol as a substrate, but there are still insufficient points to put into practical use such technology, so these points should be supplemented.

### RELATED ART LITERATURE

### Patent Literature

Korean Patent Laid-Open Patent Publication 10-2016-0028314 “Method for producing citric acid using microorganisms of the genus Aspergillus mutated by methanol”.

### Non-Patent Literature

M. C. Maldonado et al., World Journal of Microbiology and Biotechnology 9, 202-204 (1993)
Lei Yang et al., Aspergillus as a versatile cell factory for organic acid production, Fungal Biology Reviews 31, 33-49 (2017)

### DISCLOSURE

### Technical Problem

An objective of the present disclosure is to provide an organic acid production process using microorganisms of the genus Aspergillus (Aspergillus. sp), thereby mass-producing high-value-added organic acids such as oxalic acid using methanol and xylose obtained as a result of the refinement of Cl gas such as methane as substrates.

### Technical Solution

In order to achieve the above objective, the present disclosure provides a microorganisms production medium.

The microorganisms production medium is a production medium for microorganisms converting methanol to an organic acid, and the converted organic acid is oxalic acid, and the microorganism production medium includes 1% to 5% of methanol, 1% to 5% of xylose, and 0.01% to 0.05% of calcium chloride with respect to 1 L of the total medium. The production medium for microorganisms further includes potassium dihydrogen phosphate (KH₂PO₄), ammonium sulfate ((NH₄)₂SO₄), magnesium sulfate (MgSO₄), iron sulfate (FeSO₄), manganese sulfate (MnSO₄), zinc sulfate (ZnSO₄) or boric acid (H₃BO₃).

Another aspect of the present disclosure to achieve the above objective is to provide a growth medium for microorganisms.

The growth medium for microorganisms is a growth medium for microorganisms converting methanol to an organic acid, the converted organic acid is oxalic acid, and the growth medium for microorganisms includes two or more from the group consisting of 3 to 8% of glucose, 0.1 to 0.5% of yeast extract, and 1 to 5% of methanol with respect to 1 L of the total medium. The growth medium for microorganisms further includes potassium dihydrogen phosphate (KH₂PO₄), ammonium nitrate (NH₄NO₃), or magnesium sulfate (MgSO₄).

The other aspect of the present disclosure to achieve the above objective is to provide an organic acid production process using a microorganism converting methanol to an organic acid.

The organic acid production process includes: preparing microorganisms of the genus Aspergillus capable of spore formation in a medium containing 2% to 5% methanol; primary culturing the microorganisms in a growth medium for microorganisms; secondary culturing of the first cultured microorganisms in a production medium for microorganisms; and extracting the organic acid from the second cultured microorganisms and the production medium for microorganisms, in which the growth medium for microorganisms includes two or more from the group consisting of 3% to 8% of glucose, 0.1% to 0.5% of yeast extract, and 1 to 5% of methanol with respect to 1 L of the total medium. The production medium for microorganisms includes 1% to 5% of methanol, 1% to 5% of xylose, and 0.01 to 0.05% of calcium chloride with respect to 1 L of the total medium, and the organic acid is oxalic acid.

Preferably, the primary culturing may be cultured for 6 to 48 hours, and the secondary culturing may be cultured for 2 to 14 days.

The present disclosure to achieve the above objective also provides a production medium for microorganisms converting methanol to an organic acid, in which the converted organic acid is oxalic acid. The production medium for microorganisms includes 1% to 5% of methanol, 1% to 6% of xylose, and 1% to 5% of calcium chloride with respect to 1 L of the total medium. Another aspect of the present disclosure provides a production medium for microorganisms further including potassium dihydrogen phosphate (KH₂PO₄), sodium hydrogen phosphate (Na₂HPO₄), ammonium sulfate ((NH₄)₂SO₄), magnesium sulfate (MgSO₄.7H₂O) iron sulfate (FeSO₄.7H₂O), manganese sulfate (MnSO₄.5H₂O), zinc sulfate (ZnSO₄.7H₂O), or boric acid (H₃BO₃).

Another aspect of the present disclosure provides a growth medium for microorganisms converting methanol to an organic acid, in which the converted organic acid is oxalic acid. The growth medium for microorganisms includes 3% to 8% of xylose, 0.1% to 0.5% of yeast extract with respect to 1 L of the total medium, and the growth medium for microorganisms further includes potassium dihydrogen phosphate (KH₂PO₄), sodium phosphate (Na₂HPO₄), ammonium nitrate (NH₄NO₃), or magnesium sulfate (MgSO₄.7H₂O).

Still another aspect of the present disclosure provides an organic acid production process using microorganisms, the organic acid production process includes: preparing microorganisms of the genus Aspergillus capable of spore formation in a medium containing 2% to 5% methanol; primary culturing the microorganisms in a growth medium for microorganisms; secondary culturing of the first cultured microorganisms in a production medium for microorganisms; and extracting the organic acid from the second cultured microorganisms and the production medium for microorganisms.

Preferably, the primary culturing may be cultured for 24 to 48 hours, and the secondary culturing may be cultured for 2 to 8 days.

### Advantageous Effects

According to the present disclosure, provided is a medium composition of microorganisms converting methanol to an organic acid and the organic acid production process of microorganisms converting methanol to an organic acid, thereby improving methanol consumption and organic acid production to a maximum value.

### DESCRIPTION OF DRAWINGS

FIG. 1 is an image showing the oxalic acid production and growth form of microorganisms cultured according to an embodiment of the present disclosure;

FIG. 2 is another medium composition test result for comparison with the medium composition used in an embodiment of the present disclosure;

FIG. 3 is an image showing the metabolic process of the microorganisms used in an embodiment of the present disclosure;

FIG. 4 is a cultured image and WCW measurement results of microorganisms confirmed according to an experimental example of the present disclosure;

FIG. 5 is the xylose consumption confirmed according to an experimental example of the present disclosure;

FIG. 6 is the methanol consumption confirmed according to an experimental example of the present disclosure;

FIG. 7 is a cultured image of microorganisms cultured according to an embodiment of the present disclosure;

FIGS. 8 and 9 are the oxalic acid production confirmed according to an experimental example of the present disclosure;

FIG. 10 shows a mechanism for generating formaldehyde from methanol by alcohol dehydrogenase and an absorbance graph of NADH confirmed according to an experimental example of the present disclosure;

FIG. 11 is a graph showing the enzyme activity of alcohol dehydrogenase confirmed according to an experimental example of the present disclosure;

FIG. 12 is a graph showing the consumption of methanol and generation of oxalic acid when 5% methanol-resistant A. niger strain cultured in a PDA medium having 5% methanol in a production medium (excluding xylose in the production medium of Example 1) is inoculated according to an experimental example of the present disclosure; and

FIGS. 13 and 14 are graphs showing the consumption of xylose and methanol when 5% methanol-resistant A. niger strain is inoculated into the production medium of Example 1 confirmed according to an experimental example of the present disclosure, FIG. 13 shows the Xyl-Xyl experimental group, and FIG. 14 shows the Glc-Xyl experimental group.

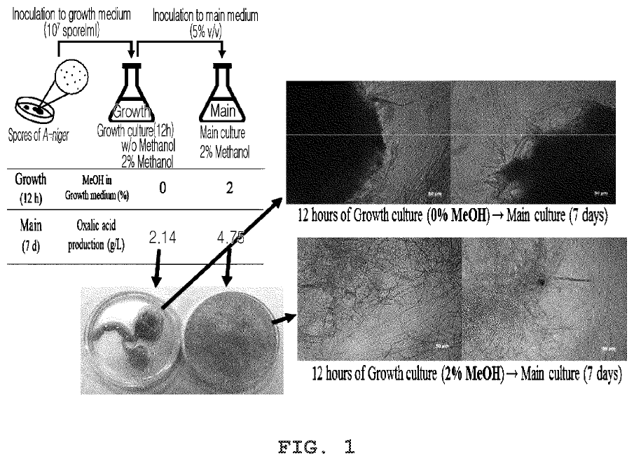

### BEST MODE

Conventionally, most organic acids have been produced by chemical synthesis. Recently, the production of organic acids by microorganisms fermentation has attracted attention due to environmental regulations, microorganisms culture technology, and the development of genetic engineering technology.

Therefore, the present disclosure is to provide a development of Aspergillus sp. strains obtained methanol resistance through evolutionary mutagenesis, which consumes methanol, low purity and low value-added substance converted from methane, as a carbon source to produce organic acids, a method for increasing methanol consumption and organic acid production through optimization of culture conditions, and various uses thereof.

In the case of oxalic acid, which is mainly produced by strain, it is a raw material compound used in a variety of pharmaceutical, paper, chemical, and food industries and is manufactured by chemical synthesis. These oxalic acids need to be manufactured by eco-friendly biological methods and may provide environmental and economic benefits through production through fungal strains.

In particular, the filamentous fungus, Aspergillus niger of the genus Aspergillus sp., may be used to produce commercial enzymes, food ingredients, pharmaceuticals, and organic acids. Biological production of these organic acids is a promising approach to obtaining building block chemicals as a renewable waste carbon source.

To this end, the use of methanol in the genus Aspergillus was confirmed at first. A methanol medium was designed and cultured to confirm the use of methanol in the Aspergillus genus, a medium based on the methanol metabolic pathway of the genus Aspergillus was designed and cultured, and methanol utilization was measured based on the minimal nutrient medium of the genus Aspergillus.

In addition, it was confirmed whether the final target product was produced through the use of methanol in the genus Aspergillus. To this end, organic acid production based on the minimal nutrient medium was measured, the organic acid production results were compared by culture condition, the organic acid production results affected by the initial spore inoculation concentration were confirmed, and the organic acid production results affected by the methanol concentration were confirmed. By confirming the result of the increase of oxalic acid according to the xylose increase condition and the result of organic acid production in the production medium (main) through the growth medium (seed), the production medium for microorganisms, the growth medium for microorganisms, and the organic acid production process that can achieve the above objective are provided.

Hereinafter, the present disclosure will be described in detail as follows.

According to one aspect of the present disclosure, a production medium for microorganisms is provided.

The microorganisms production medium is a production medium for microorganisms converting methanol to an organic acid, the microorganism is of the genus Aspergillus, the converted organic acid is oxalic acid, and the microorganism production medium includes 1% to 5% of methanol, 1% to 5% of xylose, and 0.01% to 0.05% of calcium chloride with respect to 1 L of the total medium. The production medium for microorganisms further includes potassium dihydrogen phosphate (KH₂PO₄), ammonium sulfate ((NH₄)₂SO₄), magnesium sulfate (MgSO₄), iron sulfate (FeSO₄), manganese sulfate (MnSO₄), zinc sulfate (ZnSO₄), or boric acid (H₃BO₃). The strain used for the production medium is a strain that has become resistant to a 5% methanol medium through evolutionary mutagenesis, so sufficient methanol supply is required, and a sufficient amount of xylose helps in organic acid production, and calcium ions (Ca²⁺) are an essential component for mycelial growth and spore formation, so sufficient supplying is required.

According to another aspect of the present disclosure, a growth medium for microorganisms is provided.

The growth medium for microorganisms is a growth medium for microorganisms converting methanol to an organic acid, the microorganism is of the genus Aspergillus, the converted organic acid is oxalic acid, and the growth medium for microorganisms includes two or more from the group consisting of 3 to 8% of glucose, 0.1 to 0.5% of yeast extract, and 1 to 5% of methanol with respect to 1 L of the total medium. The growth medium for microorganisms further includes potassium dihydrogen phosphate (KH₂PO₄), ammonium nitrate (NH₄NO₃), or magnesium sulfate (MgSO₄).

Preferably, the production medium and the growth medium have a composition ratio, as shown in Table 1 below.

TABLE 1

| | | Growth | Production |
|---|---|---|---|
| Components | /L | medium | medium |
| Glucose | g | 55 | |
| Yeast extract | g | 2 | |
| Methanol | g | 0.20 | 20 |
| Xylose | g | | 20 |
| CaCl₂ | mg | | 20 |
| KH₂PO₄ | g | 0.02 | 4 |
| (NH₄)₂SO₄ | g | | 2 |
| NH₄NO₃ | g | 6 | |
| MgSO₄•7H₂O | g | 4 | 0.2 |
| FeSO₄•7H₂O | mg | | 10 |
| MnSO₄•5H₂O | ug | | 10 |
| ZnSO₄•7H₂O | ug | | 20 |
| H₃BO₃ | ug | | 10 |

The still another aspect of the present disclosure to achieve the above objective is to provide an organic acid production process using a microorganism converting methanol to an organic acid.

Still another aspect is to provide a production process. The organic acid production process includes: preparing microorganisms of the genus Aspergillus capable of spore formation in a medium containing 2% to 5% methanol; primary culturing the microorganisms in a growth medium for microorganisms; secondary culturing of the first cultured microorganisms in a production medium for microorganisms; and extracting the organic acid from the second cultured microorganisms and the production medium for microorganisms, in which the growth medium for microorganisms includes two or more from the group consisting of 3% to 8% of glucose, 0.1% to 0.5% of yeast extract, and 1 to 5% of methanol with respect to 1 L of the total medium. The production medium for microorganisms includes 1% to 5% of methanol, 1% to 5% of xylose, and 0.01% to 0.05% of calcium chloride with respect to 1 L of the total medium and the organic acid is oxalic acid.

Preferably, the primary culturing may be cultured for 6 to 48 hours, and the secondary culturing may be cultured for 2 to 14 days.

For the design of a medium containing methanol (M. C. Maldonado et al., World Journal of Microbiology and Biotechnology 9, 202-204 (1993)) was referred. Aspergillus niger produces spores asexually, and the spores form stems, reproduce in the form of conidia, and can grow in a wide pH range.

As described above, it is necessary to constantly adjust the concentration of spores inoculated for each flask during incubation, and for simple spore concentration measurement, a linear relationship graph between OD550 value and spore concentration measured with a hemocytometer is prepared and is used to predict the concentration of spores using only the value.

When only methanol is present as a carbon source, there is a document that A. niger does not consume methanol, so another type of carbon source (sugar) consumed together with methanol is additionally supplied. In order to measure methanol consumption according to the type of sugar, referring to related papers, the following 4 types of medium Czapek Dox Agar, T. Lignorum, Pectin-based, and Xylose-based medium are designed and cultured.

The composition of the four types of media is shown in Table 2 below.

TABLE 2

| | | | | ²⁾Pectin- | ²⁾Xylose- |
|---|---|---|---|---|---|
| | | Czapek Dox | ¹⁾T. Lignorum | based | based |
| Components | /L | Agar + MeOH | medium | medium | medium |
| Sucrose | g | 30 | | | |
| Glucose | g | | 12 | | |
| Pectin | g | | | 12 | |
| Xylose | g | | | | 12 |
| Methanol | ml | 50 | 5 | 1 | 1 |
| K₂HPO₄ | g | 1 | 0.5 | | |
| KH₂PO₄ | g | | 0.5 | 4 | 4 |
| KCl | g | 0.5 | | | |
| CaCl₂ | ug | | 20 | 0.01 | 0.01 |
| MgSO₄•7H₂O | g | 0.5 | 0.1 | 0.2 | 0.2 |
| CuSO₄•5H₂O | ug | | 20 | | |
| MnSO₄•5H₂O | ug | | 20 | 10 | 10 |
| ZnSO₄•7H₂O | ug | | 20 | 70 | 70 |
| FeSO₄•7H₂O | g | 0.01 | | 0.01 | 0.01 |
| NaNO₃ | g | 3 | | | |
| NH₄NO₃ | g | | 2 | | |
| (NH₄)₂SO₄ | g | | | 2 | 2 |
| H₃BO₃ | ug | | | 10 | 10 |

¹⁾Rowen Tye & Andrew Willetts, Applied and Environmental Microbiology, 75761 (1977)
²⁾M. C. Maldonado et al., World Journal of Microbiology and Biotechnology 9, 202-204 (1993)

At this time, the previously developed methanol resistance/conversion A. niger is cultured in potato dextrose agar (PDA) medium containing 5% methanol, and only the spores are separated, the number of bacteria is measured, and then inoculated in a liquid medium and cultured.

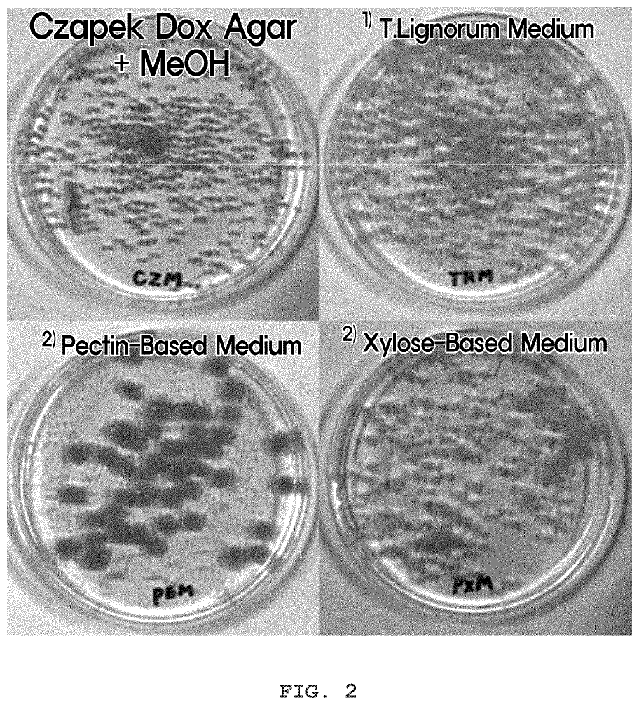

As a result of culture confirmation, the form of A. niger was different depending on the concentration of methanol and the type of sugar, and the experimental result image is shown in FIG. 2 to confirm the growth form of A. niger according to the concentration of methanol and the type of sugar.

Referring to Table 3 below, Czapek Dox medium and T. Lignorum medium consumed little methanol, and in the Pectin medium, it was difficult to confirm methanol consumption because of methanol derived from pectin. Therefore, we decided to check the methanol consumption with a Xylose-based medium.

TABLE 3

| | | | ²⁾Pectin- | ²⁾Xylose- |
|---|---|---|---|---|
| | Czapek Dox | ¹⁾T. Lignorum | based | based |
| | Agar + MeOH | medium | medium | medium |
| MeOH(day 0) (g/L) | 38.62 | 4.13 | 0.8 | 0.77 |
| MeOH(day 7) (g/L) | 36.62 ± 0.11 | 4.20 ± 0.84 | 1.26 ± 0.07 | 0.71 ± 0.00 |

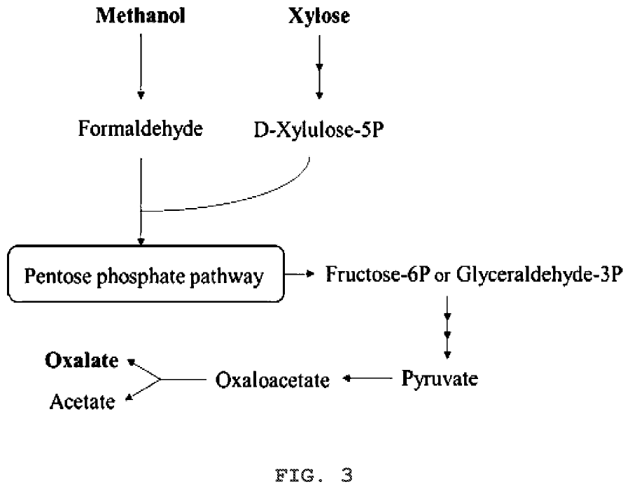

As described above, it was intended to establish a medium based on the xylose-based medium but based on the methanol metabolic pathway of the genus Aspergillus. For this, the methanol metabolic pathway of Aspergillus niger is shown in FIG. 3.

Referring to FIG. 3, in the methanol metabolic pathway of Aspergillus niger, methanol is converted to formaldehyde, and xylose is converted to xylulose-5-phosphate. Afterwards, it can be confirmed that formaldehyde and xylulose-5-phosphate are metabolized by being converted to glycerone (dihydroxyacetone) by dihydroxyacetone synthase (DAS).

This study aims to establish and confirm a minimal nutrient medium using xylose and methanol as carbon sources by referring to the papers related to the methanol metabolic pathway of Aspergillus niger.

The present disclosure also provides a production medium for microorganisms converting methanol to an organic acid, in which the converted organic acid is oxalic acid. The production medium for microorganisms includes 1% to 5% of methanol, 1% to 6% of xylose, and 1% to 5% of calcium chloride with respect to 1 L of the total medium. Provided is a production medium for microorganisms further including potassium dihydrogen phosphate (KH₂PO₄), sodium hydrogen phosphate (Na₂HPO₄), ammonium sulfate ((NH₄)₂SO₄), magnesium sulfate (MgSO₄.7H₂O), iron sulfate (FeSO₄.7H₂O), manganese sulfate (MnSO₄.5H₂O) zinc sulfate (ZnSO₄.7H₂O), or boric acid (H₃BO₃).

Provided is a growth medium for microorganisms converting methanol to an organic acid, in which the converted organic acid is oxalic acid. The growth medium for microorganisms includes 3% to 8% of xylose, 0.1% to 0.5% of yeast extract with respect to 1 L of the total medium, and the growth medium for microorganisms further includes potassium dihydrogen phosphate (KH₂PO₄), sodium phosphate (Na₂HPO₄), ammonium nitrate (NH₄NO₃), or magnesium sulfate (MgSO₄.7H₂O).

Provided is an organic acid production process using microorganisms. The organic acid production process includes: preparing microorganisms of the genus Aspergillus capable of spore formation in a medium containing 2% to 5% methanol; primary culturing the microorganisms in a growth medium for microorganisms; secondary culturing of the first cultured microorganisms in a production medium for microorganisms; and extracting the organic acid from the second cultured microorganisms and the production medium for microorganisms. The primary culturing may be cultured for 24 to 48 hours, and the secondary culturing may be cultured for 2 to 8 days.

Hereinafter, the present disclosure will be described in more detail through examples. These examples are only for illustrating the present disclosure, and it will be apparent to those of ordinary skilled in the art that the scope of the present disclosure is not to be construed as being limited by these examples.

### Preparation Example 1

Aspergillus niger, which is resistant to 5% methanol, was inoculated into a potato dextrose agar (PDA) medium containing 5% methanol and cultured at 30° C. for 3 to 4 days.

### Example 1

Aspergillus niger prepared in Preparation Example 1 was inoculated with 10⁴ spore/ml, 10⁵ spore/ml, and 10⁶ spore/ml to production medium and cultured.

The composition of the production medium includes 20 g of methanol, 20 g of xylose, 20 mg of CaCl₂, 4 g of KH₂PO₄, 2 g of (NH₄)₂SO₄, 0.2 g of MgSO₄.7H₂O, 10 g of FeSO₄.7H₂O, 10 g of MnSO₄.5H₂O, 20 g of ZnSO₄.7H₂O, and 10 g of H3BO3 per 1 L of the total medium.

### Example 2

After preparing a medium in which the content of methanol is adjusted to be 0.5%, 1%, 2%, and 4%, Aspergillus niger prepared in Preparation Example 1 is inoculated at a concentration of 10⁷ spore/ml into each medium in which the content of methanol is adjusted and cultured.

### Example 3

Prepare a production medium in which the composition was partially changed to 0.2% of methanol and 50 g of xylose, and Aspergillus niger prepared in Preparation Example 1 was inoculated into a medium in which the composition was partially changed at a concentration of 10⁷ spore/ml and incubated for 14 days.

### Example 4

The Aspergillus niger prepared in Preparation Example 1 is inoculated into a growth medium at a concentration of 10⁷ spore/ml and first cultured for 12 hours, and the first cultured Aspergillus niger culture solution is inoculated into a production medium to 5% (v/v) of the production medium and second cultured.

The growth medium composition includes 55 g of glucose, 2 g of yeast extract, 0.02 g of KH₂PO₄, 6 g of NH₄NO₃, and 4 g of MgSO₄.7H₂O with respect to 1 L of the total medium.

### Example 5

It is performed in the same manner as in Example 4, but using a medium containing 20 g of methanol for 1 L of the growth medium to be first cultured.

### Experimental Example 1

The Aspergillus niger cultured in Example 1 is measured by a wet cell weight method (WCW) in order to quickly identify the growth profile. In the case of fungus, the growth profile cannot be confirmed by OD because the mycelium aggregates with each other frequently.

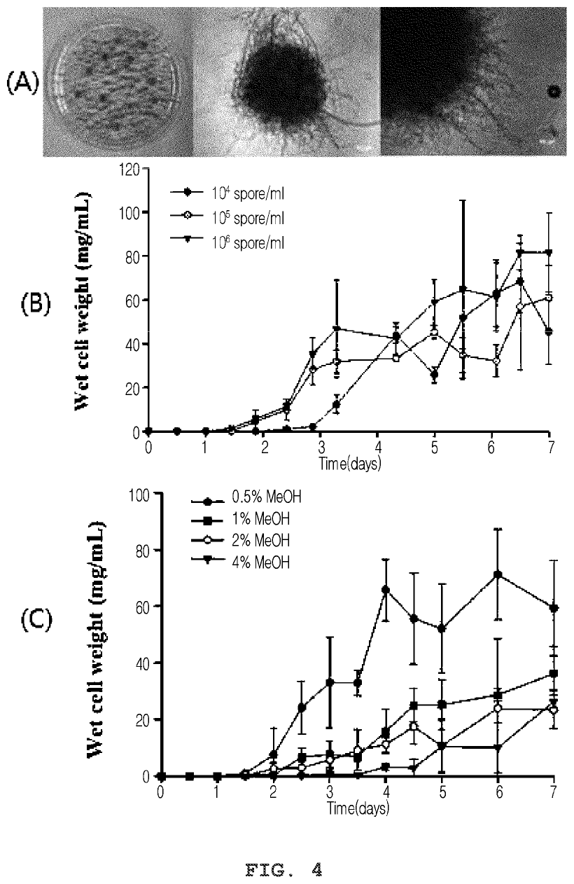

In the improved production medium, a culture photograph of A. niger (methanol 2%, initial spore concentration 94/ml) and results are shown in FIG. 4. Referring to FIG. 4A of FIG. 4, a cultured image of A. niger in the improved production medium may be confirmed, and a culture result of A. niger according to an initial spore inoculation concentration may be confirmed in FIG. 4B and growth profile according to the concentration of methanol in the medium may be confirmed in FIG. 4C.

Referring to FIG. 4B, when the initial spore inoculation concentration (methanol 2%) is changed and cultured, it may be seen that the initial growth point is increased in the 10⁵ pore/ml and 10⁶ pore/ml inoculation flask.

Referring to FIG. 4C, it may be seen that a growth profile (initial pore concentration of 10⁵ spore/ml) is different according to a methanol concentration in a medium, and it may be seen that the wet cell weight is the highest in a 0.5% methanol-containing medium.

It is necessary to check the growth profile of Aspergillus niger in the improved production medium designed as described above to determine whether organic acid comes from exponential growth so that a culture strategy can be established when cultivated in a fermentor in the future.

### Experimental Example 2

LC quantitative analysis was performed to determine the amount of xylose consumed according to the methanol concentration in the medium of Aspergillus niger cultured in Example 2 above. The analysis was performed using RID (refractive index detector) as a detector, further confirming the methanol consumption of A. niger in xylose and methanol (2%) medium.

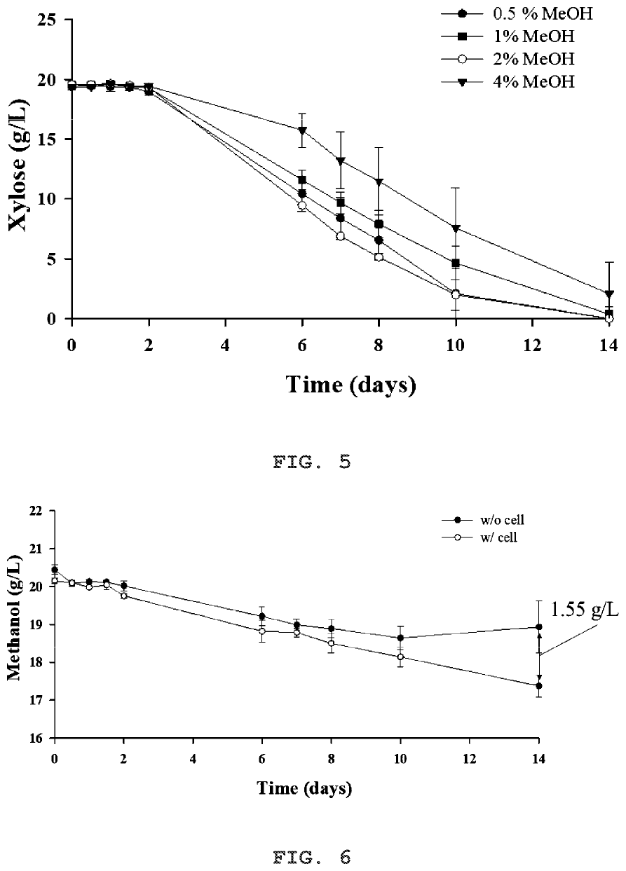

The results of measuring the xylose consumption of A. niger according to the methanol content (0.5, 1, 2, 4%) in the production medium are shown in FIG. 5, and the methanol consumption pattern of A. niger in the methanol (2%) medium is shown in FIG. 6. Referring to FIG. 6, it can be seen that 1.55 g/L of consumption appears after 14 days of culture in a medium containing 2% methanol.

### Experimental Example 3

Confirm the organic acid production result of Example 3 with a UV detector.

The organic acid production results are shown in Table 4 below.

Referring to Table 4 below, after 14 days of culture in a high concentration of xylose (50 g/L) and 0.2% methanol medium, the production of 6.55 g/L of oxalic acid was confirmed, and a maximum amount of production was 6.88 g/L.

TABLE 4
Oxalic acid

| | |
|---|---|
| Start(day 0) | 0 |
| Final(day 14) | 6.55 ± 0.31 |

In the future, if the growth medium conditions are introduced, and xylose is increased to 50 g/L, the production of oxalic acid is expected to increase further if the A. niger strain is cultured in a loosed form.

### Experimental Example 4

Methanol consumption and xylose consumption of Aspergillus niger cultured in Examples 3 and 4 were measured.

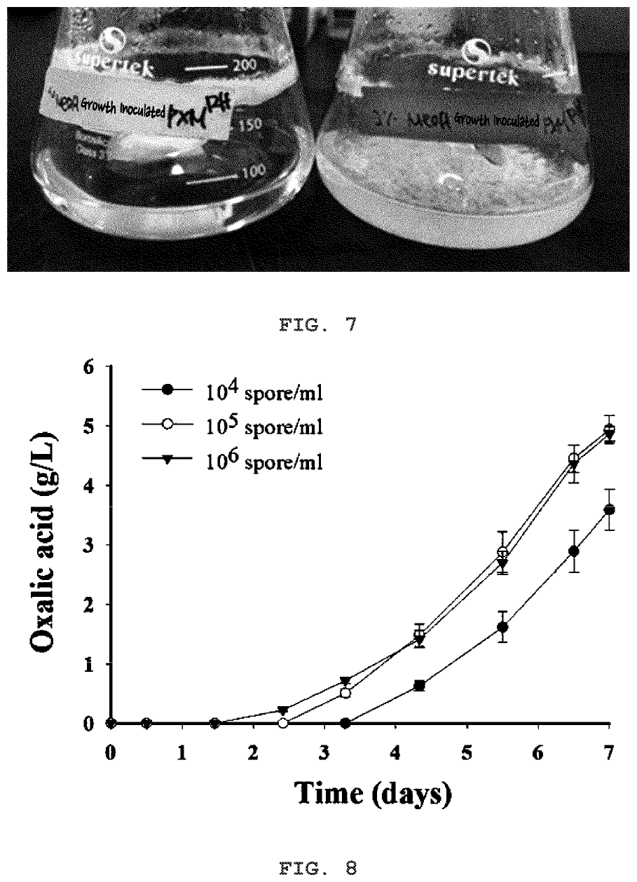

The methanol consumption results of Aspergillus niger cultured in Examples 3 and 4 are shown in Tables 5 and 6 below, and images showing the growth patterns of Examples 3 and 4 are shown in FIG. 7.

When transferred to the production medium through the growth medium as in Examples 3 and 4, more fungal cell mass than when inoculated into the production medium without going through the growth medium can be secured and grown in the production medium, and a growth medium process is required for the mycelium to spread and grow without agglomeration.

However, referring to FIG. 7, as in Examples 4 and 5, another growth form of A. niger was observed when cultured by inoculating A. niger cultured in a production medium containing 2% methanol depending on the presence or absence of methanol in the growth culture medium. The left side of FIG. 7 is an image showing that when secondary culture is performed using the seed of Example 4 cultured in a growth medium without methanol, the fungus agglomerates and grows, and the picture on the right side of FIG. 6 is an image showing that when the secondary culture is performed using the seed of Example 5 cultured in a growth medium containing methanol and the fungus spreads and grows.

TABLE 5

| | | Seed (Growth medium) | | | | | | | |
|---|---|---|---|---|---|---|---|---|---|
| | | Main phagocytosis time | | | | | | | |
| | | 12 h | | 24 h | | 48 h | | 72 h | |
| | | MeOH in the growth medium (g/L) | | | | | | | |
| | | 0 | 20 | 0 | 20 | 0 | 20 | 0 | 20 |
| Main | MeOH | 11.39 | 15.31 | 18.02 | 18.80 | 18.09 | 18.42 | 18.13 | 18.39 |
| (Production | (day 0) (g/L) | 10.90 | 11.43 | 17.08 | 16.24 | 15.80 | 16.51 | 15.93 | 17.20 |
| medium) | MeOH | 0.48 | 3.88 | 0.94 | 2.56 | 2.29 | 1.91 | 2.20 | 1.19 |
| | (day 7) (g/L) | | | | | | | | |
| | *MeOH | — | 2.43 | — | 1.11 | 0.84 | 0.46 | 0.75 | — |
| | Consumption | | | | | | | | |
| | (g/L) | | | | | | | | |

*Excluding evaporation: 7 days, 1.45 g/L

TABLE 6

| | | Seed (Growth medium) | | | | | | | |
|---|---|---|---|---|---|---|---|---|---|
| | | Main phagocytosis time | | | | | | | |
| | | 12 h | | 24 h | | 48 h | | 72 h | |
| | | MeOH in the growth medium (g/L) | | | | | | | |
| | | 0 | 20 | 0 | 20 | 0 | 20 | 0 | 20 |
| Main | Xylose | 17.82 | 17.37 | 17.67 | 17.49 | 16.53 | 18.41 | 16.55 | 18.60 |
| (Production | (day 0) (g/L) | | | | | | | | |
| medium) | Xylose | 6.85 | 0.00 | 3.55 | 0.12 | 1.92 | 4.76 | 3.32 | 5.78 |
| | (day 7)( g/L) | | | | | | | | |
| | Xylose | 10.97 | 17.37 | 14.12 | 17.37 | 14.61 | 13.65 | 13.23 | 12.82 |
| | Consumption | | | | | | | | |
| | (g/L) | | | | | | | | |

Referring to Tables 5, 6, and 7 above, after 12 hours of initial incubation in a growth medium containing glucose and nutrients, when inoculated into a xylose and methanol (2%) medium, if the growth medium contains methanol, the A. niger strain is cultured in the loose form. It can be seen that the consumption of methanol (2.43 g/L) and xylose (17.36 g/L) was increased when cultured in a loose form than when cultured in an aggregated form.

### Experimental Example 5

Confirm the organic acid production results of Examples 1 and 2 with a UV detector.

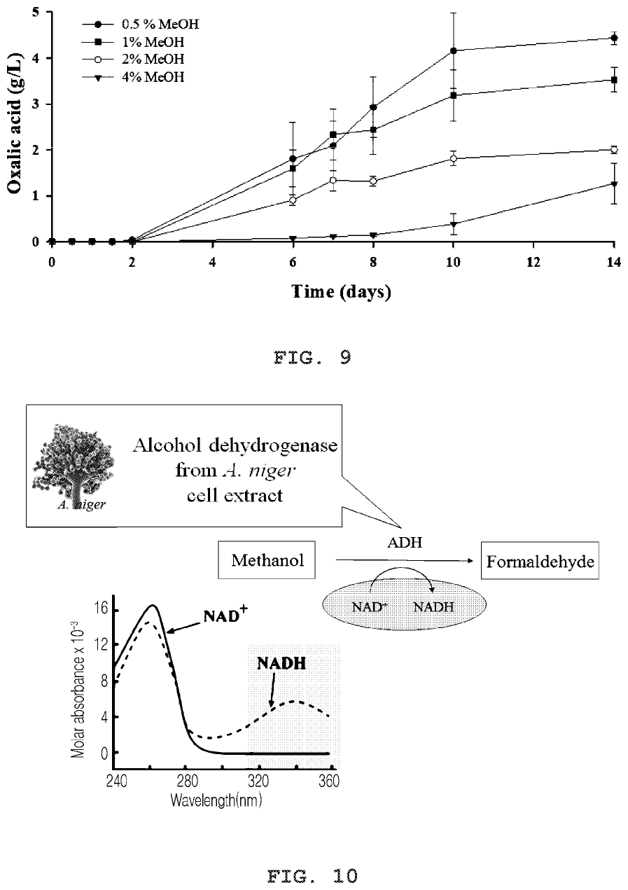

The organic acid production result of Example 1 is shown in FIG. 8, and the organic acid production result of Example 2 is shown in FIG. 9.

Referring to FIG. 8, the organic acid production results affected by the initial spore inoculation concentration can be confirmed, and it can be confirmed that about 4.9 g/L of oxalic acid is produced when inoculated at 10⁵ spore/ml and 10⁶ spore/ml concentrations. Based on this, it can be seen that the inoculation concentration of 10⁵ spore/ml is suitable when considering in terms of organic acid production yield.

Referring to FIG. 9, the organic acid production result affected by the methanol concentration can be confirmed, and it can be confirmed that about 4.5 g/L of oxalic acid is produced in a 0.5% methanol medium. In FIG. 9, it can be seen that the production amount of oxalic acid decreases as the methanol concentration increases beyond 0.5%.

### Experimental Example 6

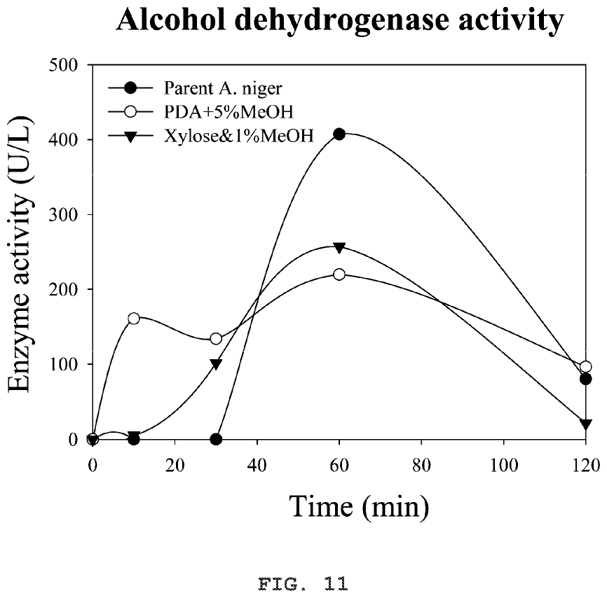

It is known that methanol is converted to formaldehyde by alcohol dehydrogenase (ADH), and NAD⁺ is converted to NADH in the process. In this case, the presence of ADH in the A. niger cell extract can be confirmed by the presence of the generated NADH, and the resulting NADH can be confirmed by the absorbance values measured at wavelengths around 340 nm (FIG. 10).

There has been a debate whether oxalic acid, which is an organic acid generated when A. niger is inoculated into the production medium of Example 1, including methanol and xylose, is produced only from xylose instead of methanol. Therefore, the following experiment was performed to confirm that oxalic acid was produced by ADH in A. niger from methanol. First, Parent A. niger, which is not resistant to methanol, was cultured in a potato dextrose agar (PDA) medium, and the cultured strain was inoculated into a test tube containing methanol and NAD+. Second, A. niger strain resistant to 5% methanol cultured in a PDA medium with 5% methanol was used in the same method as the first. Third, 20 g of xylose and 10 g of methanol were contained, and the remaining components were an agar medium contained the same as the components and weight of the production medium of Example 1. A. niger strain resistant to 5% methanol cultured in the agar medium was used. As a result, the absorbance of NADH around 340 nm was measured in each experimental group, and the enzyme activity of ADH could be measured from the absorbance value of NADH through the following Formula (FIG. 11).

$$Activity = \frac{1000 \times TV \times D \times dA / dt}{ε \times V \times CF}$$

Activity: Volumetric Activity (U/L)

TV: Total volume in cuvette

D: Dilution of the cell extract

V: Volume of cell extract used

ε: Molar extinction coefficient for NADH

CF: concentration factor of cell extract (for example, if a 100 mL sample is concentrated to a 2 mL volume for the French press, then CF=50)

As a result of the experiment, the highest methanol degradation enzyme activity was shown in the parent strain, but the strain did not grow normally in the production medium in which methanol was present. It was found that the concentration of formaldehyde increased rapidly due to the rapid increase in enzyme activity of ADH, and the rapid increase in formaldehyde showed cytotoxicity, and strain did not grow normally. The enzyme activity started the fastest in the 5% methanol-resistant strain cultured in a PDA medium with 5% methanol. Since the normal growth of strains is important for the methanol-organic acid conversion process, it was judged that using 5% methanol-resistant A. niger strains cultured in a PDA medium with 5% methanol was most appropriate.

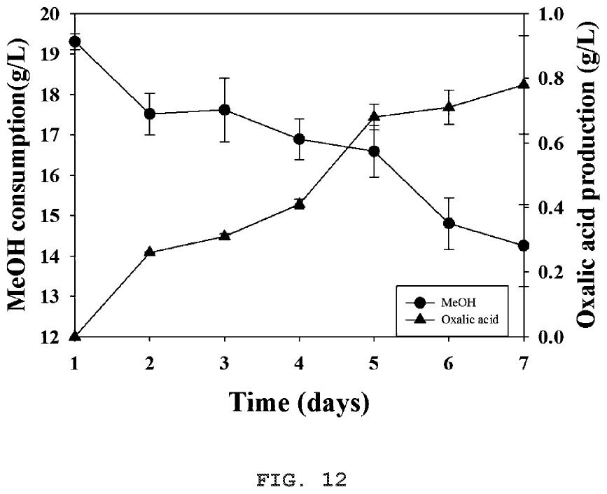

In addition, in the production medium excluding xylose (excluding xylose in the production medium of Example 1), the 5% methanol-resistant A. niger strain cultured in the PDA medium with 5% methanol was inoculated. That is, except for xylose (0 g/L), culture was performed in a medium containing only methanol as a carbon source, and as a result, methanol consumption occurred (3.5 g/L was consumed excluding natural evaporation), and it was confirmed that oxalic acid was produced (about 0.8 g/L). FIG. 12 shows that the A. niger strain resistant to 5% methanol currently used can consume methanol to produce organic acids.

### Experimental Example 7. Scale Up

10⁷ spores/ml of 5% methanol-resistant A. niger strain (cultured in a PDA medium with 5% methanol) was inoculated to the glucose-based growth medium (Growth medium-Glucose in Table 7), a growth medium of Example 5 containing glucose and methanol, and cultured for 30 hours. Thereafter, the cultured A. niger strains were inoculated with 5% v/v to 3 liters of the production medium (corresponding to the main medium in Table 7) in the fermenter (5 liters) and cultured for 5 days. (Glc-Xyl experimental group)

A xylose-based growth medium containing 70 g/L of xylose instead of glucose, 2 g/L of yeast extract, 2 g/L of potassium dihydrogen phosphate (KH₂PO₄), 2 g/L of sodium dihydrogen phosphate (Na₂HPO₄), 6 g/L of ammonium nitrate (NH₄NO₃), and 4 g/L of magnesium sulfate (MgSO₄.7H₂O) was designed. (Growth medium-xylose in Table 7). Thereafter, 10⁷ spores/ml of A. niger strain resistant to 5% methanol (obtained after culturing in PDA medium with 5% methanol) was inoculated to the newly designed growth medium and cultured. Then, the cultured A. niger strain was again inoculated with 5% v/v in 3 liters of the production medium (main medium in Table 7) contained in the fermenter (5 liters). (Xyl-Xyl experimental group).

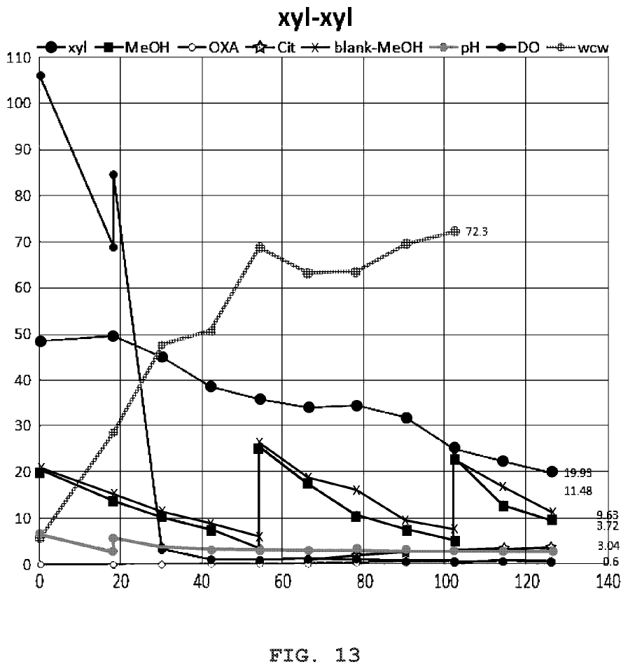

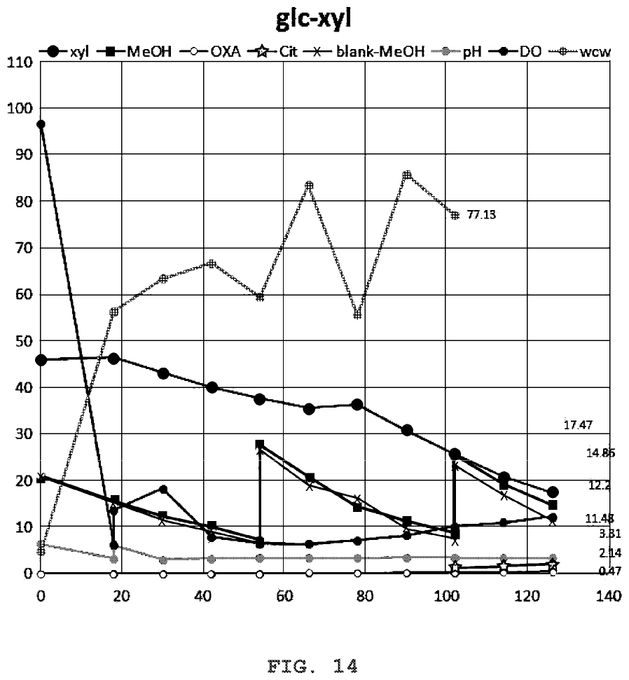

When the results shown in FIGS. 13 and 14 and Table 8 are summarized, it can be seen that in the case of the strain inoculated after culturing in the xylose-based growth medium (Xyl-Xyl experimental group), the consumption of methanol was significantly increased. (6.23 g/L excluding the natural evaporation amount of 44.99 g/L, Table 8). This is because when xylose is supplied as a carbon source instead of glucose from the growth medium, the pathway using xylose is activated from the beginning, and in the case of the pathway using xylose, it overlaps with the pathway using actual methanol except for the beginning of the pathway (FIG. 3), it seems that the pathway for consuming xylose and methanol was more activated even in the production medium.

TABLE 7

| | | Growth medium- | Growth medium- | Main |
|---|---|---|---|---|
| Components | /L | Glucose | Xylose | medium |
| Glucose | g | 55 | 0 | |
| Yeast extract | g | 2 | 2 | |
| Methanol | g | 20 | 0 | 20 |
| Xylose | g | | 70 | 50 |
| CaCl₂ | mg | | | 20 |
| KH₂PO₄ | g | 0.02 | 2 | 4 |
| Na₂HPO₄ | g | | 2 | 6 |
| (NH₄)₂SO₄ | g | | | 2 |
| NH₄NO₃ | g | 6 | 6 | |
| MgSO₄•7H₂O | g | 4 | 4 | 0.2 |
| FeSO₄•7H₂O | mg | | | 10 |
| MnSO₄•5H₂O | ug | | | 10 |
| ZnSO₄•7H₂O | ug | | | 70 |
| H₃BO₃ | ug | | | 10 |

TABLE 8

| | Xylose consumption | MeOH consumption |
|---|---|---|
| Xyl-Xyl(g/L) | 28.62 | 51.23(−6.23) |
| Glc-Xyl(g/L) | 28.66 | 42.96(+2.03) |
| Blank(g/L) | | 44.99 |

As described above, it will be apparent to those skilled in the art that such a specific technique is merely a preferred embodiment, and thus the scope of the present disclosure is not limited thereto. Therefore, it will be said that the practical scope of the present disclosure is defined by the appended claims and their equivalents.

1. A production medium for microorganisms converting methanol to an organic acid,
wherein the organic acid is oxalic acid,
the production medium comprises 1% to 5% of methanol, 1% to 5% of xylose, and 1% to 5% of calcium chloride with respect to 1 L of the medium, and
the production medium further comprises potassium dihydrogen phosphate (KH2PO4), ammonium sulfate ((NH4)2SO4), magnesium sulfate (MgSO4.7H2O), iron sulfate (FeSO4.7H2O), manganese sulfate (MnSO4.5H2O), zinc sulfate (ZnSO4.7H2O), or boric acid (H3BO3).

2. A growth medium for microorganisms converting methanol to an organic acid,
wherein the organic acid is oxalic acid,
the growth medium comprises 3% to 8% of glucose, 0.1% to 0.5% of yeast extract, and 1% to 5% of methanol with respect to 1 L of the medium, and
the growth medium further comprises potassium dihydrogen phosphate (KH₂PO₄), ammonium nitrate (NH₄NO₃), or magnesium sulfate (MgSO₄.7H₂O).

3. An organic acid production process using microorganisms converting methanol to an organic acid, the production process comprising:
Preparing, as the microorganisms, the genus Aspergillus capable of spore formation in a medium containing 2% to 5% methanol;
primary culturing the microorganism in a microorganism growth medium;
secondary culturing the primarily cultured microorganisms in a microorganism production medium; and
extracting an organic acid from the secondarily cultured microorganisms and the microorganism production medium,
wherein the microorganism growth medium comprises 3% to 8% of glucose, 0.1% to 0.5% of yeast extract, and 1% to 5% of methanol with respect to 1 L of the medium,
and the microorganism production medium comprises 1% to 5% of methanol, 1% to 5% of xylose, and 1% to 5% of calcium chloride with respect to 1 L of the medium.

4. The production process of claim 3, wherein the primary culturing is performed for 6 to 24 hours.

5. The production process of claim 3, wherein the secondary culturing is performed for 2 to 14 days.

6. A production medium for microorganisms converting methanol to an organic acid, the organic acid being oxalic acid,
wherein the production medium comprises 1% to 5% of methanol, 1% to 6% of xylose, and 1% to 5% of calcium chloride with respect to 1 L of the medium, and
the production medium further comprises potassium dihydrogen phosphate (KH₂PO₄), sodium hydrogen phosphate (Na₂HPO₄), ammonium sulfate ((NH₄)₂SO₄), magnesium sulfate (MgSO₄.7H₂O), iron sulfate (FeSO₄.7H₂O), manganese sulfate (MnSO₄.5H₂O), zinc sulfate (ZnSO₄.7H₂O), or boric acid (H₃BO₃).

7. A growth medium for microorganisms converting methanol to an organic acid, the organic acid being oxalic acid,
wherein the growth medium comprises 3% to 8% of xylose and 0.1% to 0.5% of yeast extract with respect to 1 L of the medium, and
the growth medium further comprises potassium dihydrogen phosphate (KH₂PO₄), sodium hydrogen phosphate (Na₂HPO₄), ammonium nitrate (NH₄NO₃), or magnesium sulfate (MgSO₄.7H₂O).

8. An organic acid production process using microorganisms converting methanol to an organic acid, the process comprising:
preparing the genus Aspergillus as microorganisms capable of spore formation in a medium containing 2% to 5% methanol;
primarily culturing the microorganisms in the growth medium comprising 3% to 8% of xylose and 0.1% to 0.5% of yeast extract with respect to 1 L of the medium and further comprises potassium dihydrogen phosphate (KH₂PO₄), sodium hydrogen phosphate (Na₂HPO₄), ammonium nitrate (NH₄NO₃), or magnesium sulfate (MgSO₄.7H₂O);
secondarily culturing the primarily cultured microorganisms in the production medium comprising 1% to 5% of methanol, 1% to 6% of xylose, and 1% to 5% of calcium chloride with respect to 1 L of the medium and further comprising potassium dihydrogen phosphate (KH₂PO₄), sodium hydrogen phosphate (Na₂HPO₄), ammonium sulfate ((NH₄)₂SO₄), magnesium sulfate (MgSO₄.7H₂O), iron sulfate (FeSO₄.7H₂O), manganese sulfate (MnSO₄.5H₂O), zinc sulfate (ZnSO₄.7H₂O), or boric acid (H₃BO₃); and
extracting an organic acid from the secondarily cultured microorganisms and the production medium.

9. The production process of claim 8, wherein the primarily culturing is performed for 24 to 48 hours.

10. The production process of claim 8, wherein the secondarily culturing is performed for 2 to 8 days.
